(19) United States
(12) Patent Application Publication
(10) Pub. No.: US 2014/0193857 A1
(43) Pub. Date: Jul. 10, 2014

(54) CENTRIFUGE TUBE DROPLET GENERATOR

(71) Applicant: Cypho, Inc., Long Beach, CA (US)

(72) Inventor: Mark A. Gray, Long Beach, CA (US)

(73) Assignee: Cypho, Inc., Long Beach, CA (US)

(21) Appl. No.: 14/024,145

(22) Filed: Sep. 11, 2013

Related U.S. Application Data
(60) Provisional application No. 61/700,241, filed on Sep. 12, 2012.
(60) Provisional application No. 61/734,952, filed on Dec. 7, 2012.

(51) Int. Cl. C12Q 1/68; B01L 3/00

(52) U.S. Cl. CPC C12Q 1/686; B01L 3/5021
USPC 435/91.2; 435/304.1

(57) ABSTRACT

A centrifuge tube droplet generator includes a centrifugally generated droplet system which accommodates a sample volume of below 20 microliters and below. The system has virtually no dead volume, is simple and inexpensive and can therefore be realized in a disposable unit. The system can be tailored to fit the format of standard desktop centrifuges which are ubiquitous in biological laboratories.

### CROSS-REFERENCE TO RELATED APPLICATIONS

This application claims the benefit of U.S. Provisional Application No. 61/700,241 (Attorney Docket No. 44871-703.101), filed Sep. 12, 2012, and of U.S. Provisional Application No. 61/734,952 (Attorney Docket No. 44871-703.102), filed Dec. 7, 2012, the entire contents of which are incorporated herein by reference.

### BACKGROUND OF THE INVENTION

1. Field of the Invention

The ongoing adoption of inexpensive micro-electro-mechanical systems (MEMS)-based laboratory tools has enabled rapid and highly sensitive detection of ever diminishing sample volumes. Likewise, it has spurred the need for improved microscale particle and droplet sample preparation systems.

As an example, amplifying DNA by emulsion Polymerase Chain Reaction (ePCR) is a process of amplifying DNA in microdroplets prior to loading the droplets onto a sequencing platform.

Low sample volumes are necessary for ePCR, and suitable methods for creating droplets are not available. For example, the Microdroplet Generator like other existing microdroplet devices, has internal valves and tubing that create a dead volume space. Dead volume is not suitable for ePCR (emulsion PCR), which may only comprise a fluid sample of about 20 to about 100 microliters (μls) which is about the volume of a single droplet of water.

Many droplet generation systems are available. However, these systems do not meet the cost and performance requirements of the market. Generally, droplets are made by employing high shear forces. In contrast, the invention uses a droplet forming flow. Similar to a pressure-driven microfluidic chip, the disclosed invention uses microfluidic features to create a regular flow of droplets by merging two immiscible fluids with each other such that flow instabilities create a steady stream of regularly sized droplets.

Some systems include T-junction chips. However, T-junction chips do not make the most uniform droplets. A disadvantage of some chip based microdroplet production systems is that they are impractical to decontaminate, but too expensive to throw away after one use.

2. Description of the Background Art

US Patent Publication Nos. 2012/0190032 and 2011/0086780 describe systems for generation small droplet emulsions for performing ePCR and other procedures.

### BRIEF DESCRIPTION OF THE DRAWINGS

FIG. 1 illustrates an insert tube according to the present invention placed in a sample tube.

FIGS. 2 and 3 illustrate an exploded view of the insert and sample tube of FIG. 1.

FIG. 3 illustrates an insert according to the present invention placed in a sample tube.

FIG. 4 is a cross-sectional view of the assembly of the insert and sample tube of FIG. 1.

FIG. 5 is a detailed cross-sectional view of the assembly of the insert and sample tube of FIG. 1 showing a stack of disks for generating droplets. The reservoirs in the insert for separately holding the two fluids that enter the dist stack are not shown.

FIG. 6 is an enlarged view of the disk stack mounted in the bottom of the insert tube of FIG. 1.

FIG. 7 is an enlarged view of the droplet generating disks of FIG. 6 showing a small central hole which acts as a nozzle for receiving a first fluid from a first reservoir. The two larger ports on either side of the small hole receive a second fluid from a second reservoir and direct the second fluid to a location just beneath the nozzle defined by the small hole. The first fluid flows as a narrow stream into the second fluid and together the fluids pass through a constriction which causes the fluids to form droplets in an emulsion as they flow out of the disk stack.

FIG. 8 is an exploded view of the disks that are assembled in the insert and sample tube.

FIG. 9 is a detailed view of a microfluidic face of the showing through holes which open to the first fluid reservoir behind the central portion of the face (not shown) and the second pair of fluid ports in communication with the interior of the insert that forms the second fluid reservoir. A cover (not shown) seals the microfluidic faceplate.

FIG. 10 shows a T-junction embodiment of an insert body with first and second reservoirs. The back surface of the microfluidic face and the first fluid port can be seen.

FIG. 11 is a cross-sectional view of a insert having a microfluidic face as in FIG. 9 being inserted into a sample tube.

FIG. 12 is an enlarged non-sectional view of the insert and sample tube as shown in FIG. 11.

FIG. 13 is a cross sectional view of a distal end of the T-junction insert body of FIG. 10 showing separation of the first and second reservoirs, where the first reservoir is attached perpendicularly to the back surface of the microfluidic face and the second reservoir annularly surrounds the first reservoir.

FIG. 14 is an enlarged view of the microfluidic face of the insert of FIGS. 9-13 showing the face oriented at 45 degrees relative to the longitudinal axis of the insert. The central channel of the T-junction is aligned perpendicularly to a central axis of rotation of a centrifuge which will cause the fluids to emerge from the microfluidic face aligned with the flow of fluids away from the axis of rotation.

FIGS. 15 and 16 are assembly views of an insert and sample tube having side channels which provide a second fluidic flow path.

FIGS. 17 and 18 show a further embodiment of the insert of the present invention having a capillary tube with a notch, where the capillary tube allows flow of the first fluid under centrifugal force which causes the distal end of the capillary tube to align with the first fluid flow. The notch promotes droplet generation by allowing the second fluid to periodically interrupt the first fluid flow.

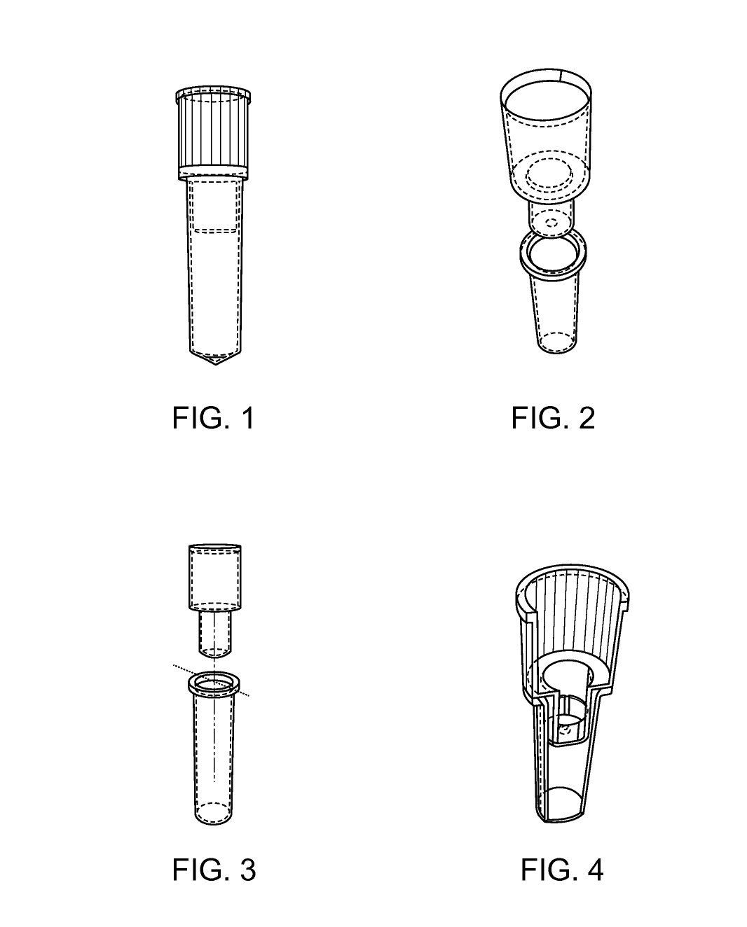

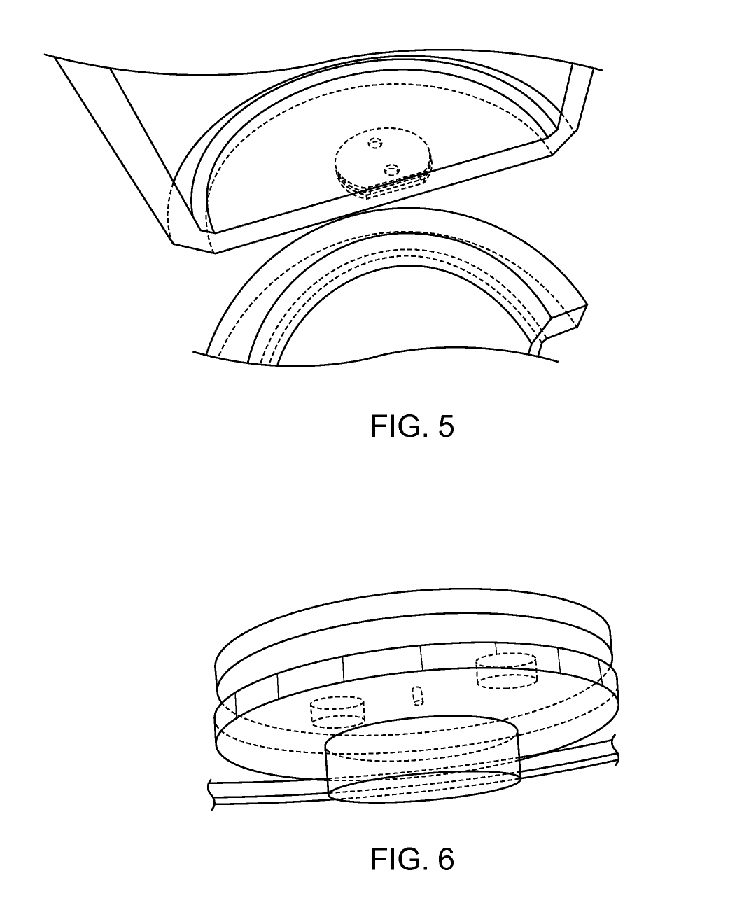

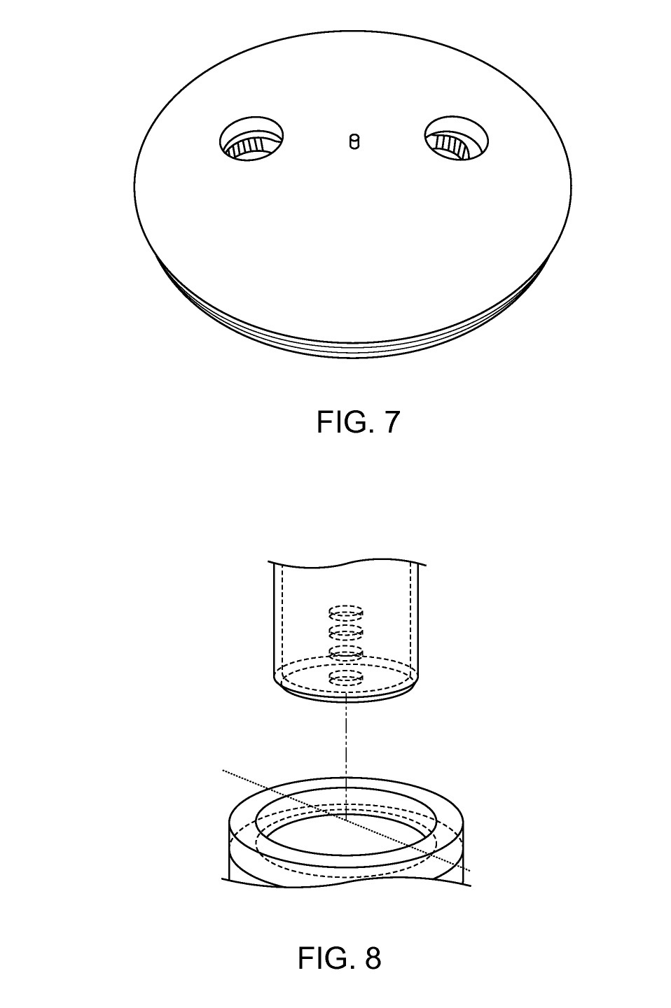

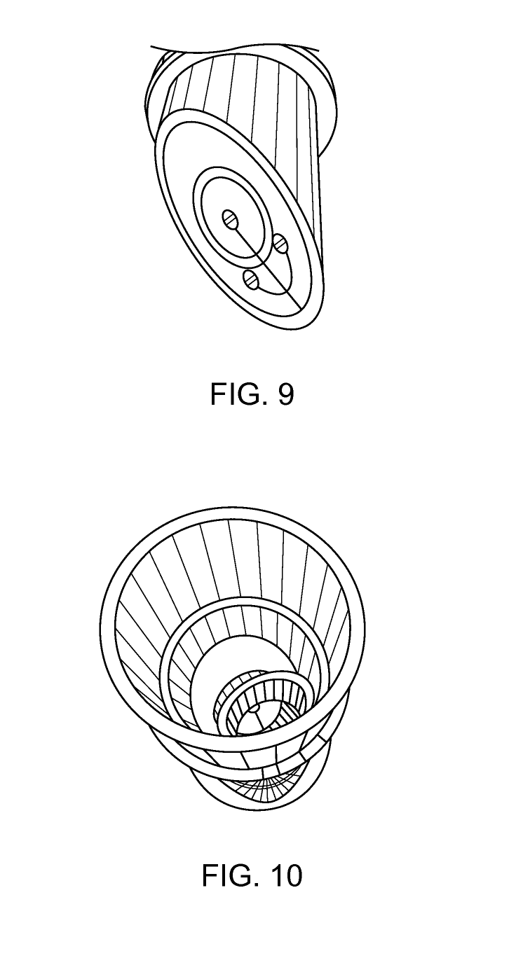

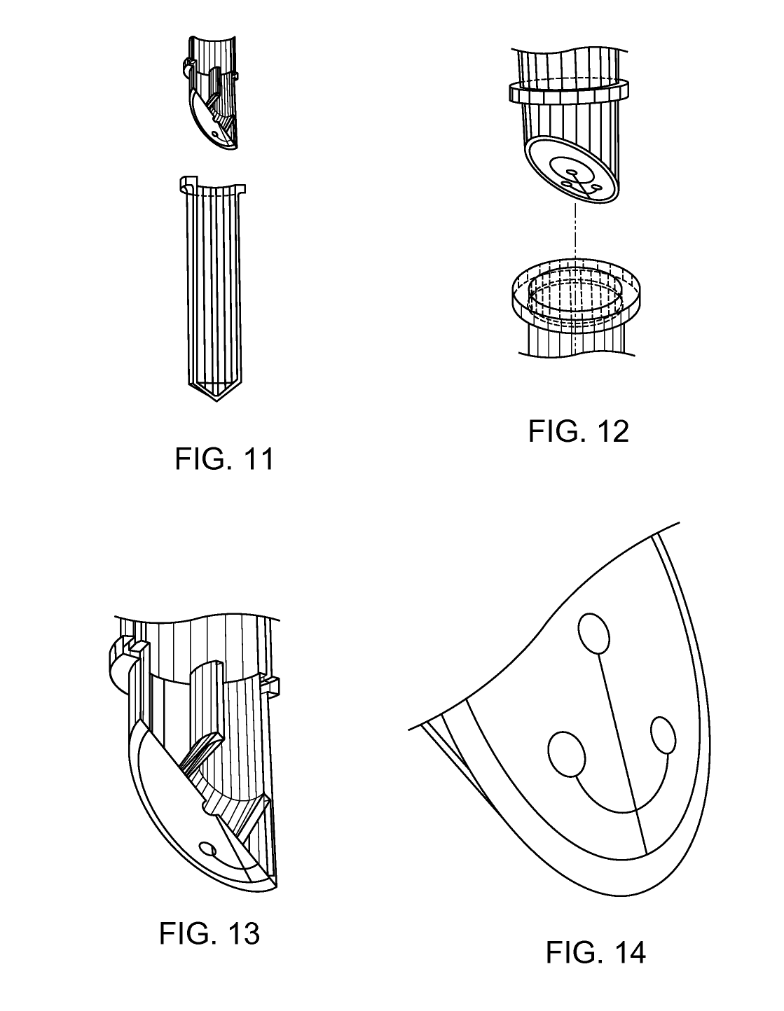

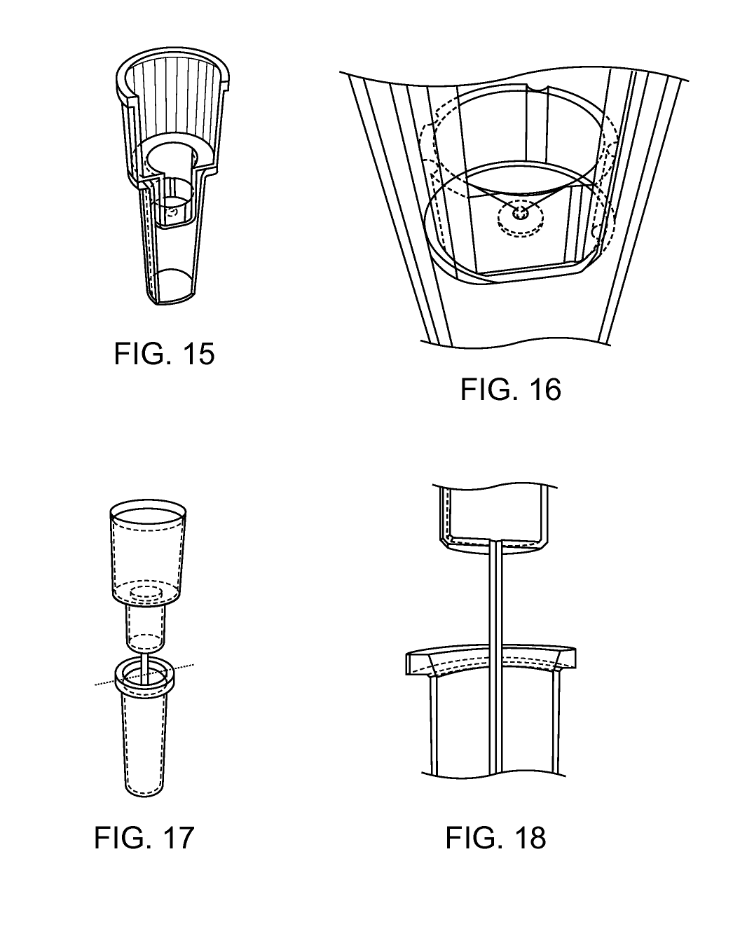

### DESCRIPTION OF THE INVENTION

The microdroplet generating systems of the present invention are particularly suitable for the processing of ePCR samples. The designs of the present invention follows certain design principles making the systems particularly suitable for an ePCR product including a small tube format to handle small sample volumes; a disposable product to minimize cross contamination; an integrated filter to minimize blockage; a clear container to facilitate loading of small samples by pipette suggests; minimal wetted surfaces to achieve a minimal dead volume; plastic molded components to provide a low cost disposable product; and use of centrifugal force without any valves or pumps and with minimal surface adhesion to achieve uniform fluid pressure.

In some embodiments of the present invention, a centrifuge tube droplet generator includes: a centrifugally generated Droplet System has the benefits of reducing sample volume to below 20 microliters; virtually no dead volume; is simple and inexpensive and can therefore be realized in a disposable unit; can be tailored to fit the format of standard desktop centrifuges, which are ubiquitous in biological laboratories.

The ePCR market is expanding rapidly. There are systems on the market that will take a liquid sample and form them into droplets, but the droplet size range is unacceptably variable. For instance, the BD (Becton Dickinson) system reports a CV (coefficient of variation) of about 50%. Such a large CV results in higher cost, more time consumed, and the loss of data.

There are few options for producing a quality emulsion for ePCR. The main reason is that there is very little fluid volume to start with. The essence of ePCR sample prep is to take about 100 microliters (equivalent to about a drop from an eyedropper) and divide that volume of fluid into about 5 million microdroplets. The purpose of dividing the fluid sample into millions of smaller droplets is to distribute and segregate the DNA molecules among a multitude of reaction chambers. For some applications, the objective is to get each DNA molecule of interest into its own droplet of PCR reagents, but not two DNA molecules into a single droplet. In that scenario, the more drops the better, but with only a small volume of starting material, e.g., 100 uL, the total number of droplets produced is limited to several million.

Embodiments of the present invention enable the generation of meaningfully monodisperse (less than 50% CV) droplet generation in a centrifuge tube format. The invention is comprised of a tube assembly that fits into the rotor of a centrifuge. Prior to centrifugation, the tube assembly contains the two or more fluids that are to be combined into droplets. Droplets are formed while the tube assembly is spun in a centrifuge. Centrifugal motion provides the motive force that drives the two or more fluids through microdroplet generating features. Those features may be described as a T-junction chip, an ejection nozzle, or other microdroplet forming microfluidic channels known to the art.

Many droplet generating features are well known to the arts and it is conceivable that they may be adapted for use with the invention. However, there are manufacturing considerations that make some droplet generating systems more preferable than others.

In general, the invention is composed of a tube insert and a tube. The tube insert contains at least two fluid reservoirs, which, during operation, are in fluid communication with a microfluidic droplet generating pathway. Such pathways are known in the art. For example, one such common droplet generating pathway is a T-junction, which operates by directing two fluids into a common channel. When two immiscible fluids are flowed into one another and into a common channel, droplets can be formed. T-junctions and other microfluidic droplet generating pathways require pressurized fluids to operate. Fluid pressure can be created using a syringe pump, a pressurized gas headspace, fluid height, or other means of providing a fluid pressure. The present invention allows for a motive force to be provided by centrifugal force.

In general, droplet generating microfluidic devices are formed using thin sheets of glass or other substrates to form the microfluidic channels including a droplet generating pathway. Such microfluidic components are collectively referred to as “chips”. To use a chip, tubes containing the liquid reagents are physically connected via ports on the chip surface to an external pressurized fluid reservoir. Droplet generation is achieved when a regular flow of reagents is flowed through the chip. In general, the more regular the flow rates and pressures, the more uniform the droplet diameter and production rate.

Generating droplets using centrifugal force is different than flowing pressurized fluid supplies through a chip. First, fluids flowing through the chip are not pressurized per se. That is, while pressurized fluids will flow equally in any direction of pressure gradient, centrifugal force is directional. In a centrifuge, fluids flow outward from the center axis of rotation. Where there is a constriction, fluids behind the constriction, such as a small nozzle or narrow channel, create a pressure behind that constriction that is functionally similar to pressurized fluids, but that does not require a closed vessel as a pressurized container. Importantly, centrifugal force is greater at distances farther away from the central axis of rotation for a given speed of rotation. This phenomena has the benefit of drawing droplets away from each other by virtue of their mass as they are created without relying on flowing fluids. A centrifugal droplet generating system was developed using a Compact Disc (CD) format and a T-junction. The present invention uses a tube format with onboard reagents that are consumed during the droplet generating process.

In the following sections and throughout this disclosure, the term “centrifugal force” is used to describe the apparent outward force that draws a rotating mass away from the center of rotation. The fluid pressures generated in a rotating centrifuge are caused by inertia as the mass of the rotating body is constantly being redirected in a curving path. While not a true force, the term “centrifugal force” is widely understood, and is used herein for clarity.

In one embodiment, the invention consists of a tube and an insert. The tube is a capture container for the fluid emerging from the insert. The insert fits into the top of the tube.

Before operation, the tube insert is filled with the two or more fluids necessary to generate an emulsion. To use the invention, the tube is spun in a centrifuge. The rotating centrifuge provides the motive force to drive the fluids contained within the insert outward from the central axis. Fluid channels direct the reagents toward and through droplet generating features. Such feature may take a number of forms, and many droplet generating fluid junctions are well known in the art. In some embodiments of the present invention, any droplet generating fluid pathway may be used provided that it is in direct fluid communication with the fluid reservoirs in the insert and the motive force is provided by centrifugal force.

The microdroplet generating features are located downstream from the fluid reservoirs in the insert and form a part of the insert body.

Reagents are added by the user or they may come shipped in the insert. Before operation, the insert contains two immiscible fluids that form an emulsion. For ePCR applications, the fluids consist of an aqueous PCR reagent mix and an oil. When the tube and insert are filled, assembled, and centrifuged, the fluids contained therein are forced outward from the center of rotation. In some instances, this equates to a downward flow of reagents within the tube insert. Standard centrifuges are made such that the tubes are actually installed at a 45 degree angle. Accordingly, the droplet generating features can be aligned in that axis. The droplet generating position of the tube is in liquid communication with the reservoirs in the upper insert. When the tube is spun at a 45 degree angle at some distance with respect to its central vertical axis, the fluids contained in the insert are forced into the droplet production area. The area can have a variety of shapes. The present disclosure includes several types of layouts but the important thing is that there are several ways to create droplets in a microfluidic chip and using a centrifuge according to embodiments of the present invention.

Centrifuges are standard laboratory instruments. The majority of all DNA purification protocols require the use of one. Desktop microcentrifuges with a rotor fixed at 45 degrees are the most common type. Other types of centrifuges are available, including larger format and swinging-bucket rotors and those are also compatible with embodiments of the present invention. It is understood that although the disclosed examples assume the use of the more common fixed angle rotor, the invention can be practiced in any centrifuge format.

In one embodiment of the invention, a glass T-junction chip is positioned downstream of two fluids flowing under centrifugal force. In this example, droplets of a first fluid are formed into the flow of a second, continuous fluid. A variation on this example is the addition of a third or more fluids. Mixing fluids in microfluidic devices is well known in the arts.

In another embodiment of the invention, a thin foil is positioned downstream of the fluid flowing under centrifugal force. The thin foil has a small diameter hole in it and as the tube is spun, the fluid behind the foil is pressed through it. An extruded flow of fluid flows into the fluid on the other side of the foil. Droplets are formed when the extruded stream of the first fluid breaks up into droplets in the second fluid.

In another embodiment, a capillary microtube is placed below a first fluid and above a second. When the tube is spun, the first fluid flows through the capillary. Droplets are formed when the first fluid flows out of the capillary into the second.

In another embodiment, a capillary tube with a small opening is placed between the two fluids. When the tube is spun, the small opening allows for the second fluid to flow into it.

In another embodiment, oil can overlay the water in a shared reservoir. The purpose of this is to increase and equalize the pressures of the two fluids during operation; the two fluids will share a head height during rotation and thus a similar pressure, regardless of reagent consumption. Fluids sharing a head height will experience a rise or reduction of fluidic pressure during rotation in proportion to the rise or reduction of that head height. In some embodiments, a smaller volume of heavier fluid is drawn from the bottom of the reservoir while the larger volume of lighter fluid is drawn from above the top of the heavier fluid. In this example, the device is designed to allow the heavier fluid to be the limiting reagent, and it runs out first. However, as the heavier fluid is removed, the head height drops. If the two reservoirs are separate, then as the water head height approaches zero, the pressure of the water would also approach zero. In some cases it is better to incorporate the water reservoir into the oil reservoir such that as the water and oil are depleted, there still remains a large amount of oil in the headspace. Even if the water runs out, there is still oil in the headspace that will provide the needed pressure to push the last of the water through the microchannels.

In another embodiment of the invention, the first reservoir can be separate from the second reservoir. This allows each of the fluids to flow independently of the other as their relative head heights are adjusted during reagent flow.

In another embodiment of the invention the microfluidic features are integral to the tube.

In another embodiment of the invention droplets are formed in a laminated structure that is connected to the fluid reservoirs. One version of the laminated structures may be drawn as a series of fluidic disks.

In another embodiment of the invention a T-junction is integral to the insert, forming the opposite surface of the bottom of the reservoirs, and the plane of the t-junction is tilted at 45 degrees from the vertical axis of a standard fixed angle rotor centrifuge.

In another embodiment of the invention, tubes are supplied with reagents sealed in the insert. Alternatively, reagents are supplied with a kit; and the insert is supplied as a component of that kit.

In another embodiment of the invention, the flow of reagents during centrifugation includes an upward portion of flow (against the flow of centrifugal force).

In another embodiment of the invention, droplets are formed by flowing reagents through a capillary tube. Droplets are formed when the first fluid, which starts at a position closer to the center of rotation, is drawn into the second fluid, located in the capture tube, during operation.

In another embodiment of the invention, microfluidic channels are not integral to the insert but are instead assembled therewith to form the requisite droplet forming features.

In another embodiment of the invention, three layers are used to form the droplet generation features. The first layer has a first small hole, which serves as a water nozzle and a second larger hole which serves to connect the oil reservoir to the second layer. The second layer connects to the oil hole and passes laterally to the area immediately under the water nozzle, where a larger expanse of the second layer is open. The third layer contains a single hole which is slightly larger than the nozzle in the first layer. The three layers function in a way that is rather like a t-junction formed vertically rather than laterally.

In another embodiment of the invention, a filter element is integrated into the insert such that particulates are removed prior to one or more of the fluids entering the flow area in order to both prevent blockage, preserve operation of the device, or to simplify one or more processing steps in an ePCR workflow.

In another embodiment of the invention, column matrix material is placed in the tube assembly. The column material would be used to combine one or more steps in an ePCR sample prep or processing workflow.

In another embodiment of the invention, the emulsion generating assembly is supplied as a part of a kit. The kit, as an example, may be for an ePCR application.

In another embodiment of the invention, a flow focusing microfluidic pathway is contained within the insert to form droplets that are smaller in diameter than the width of the channel or nozzle used in their formation.

In another embodiment of the invention, the speed of centrifugation changes over the course of a given run of the device. For example, as a fluid level is lowered during operation, the centrifuge may increase its rotational speed in order to maintain a relatively constant flow of fluids with a lessening head height. Centrifugation speed may be increased or decreased in order to affect fluid flow pressures and rates during separate phases of a sample preparation run.

In another embodiment of the invention the two separate reservoirs in the insert are formed such that the fluids exert relatively even pressure during device operation. For example, the reservoir sides containing the aqueous fluid in an ePCR sample may be considerably lower than the portion of the insert containing oil, which can then fill the chamber to overlay the aqueous sample. During operation, the two fluids are kept separate from each other, but the overlaying fluids assure that the two fluids are relatively equally pressurized. In a further example, the fluids and insert may be so arranged that the aqueous material forms a short head height relative to the non-aqueous phase, such that when the device is operated, the aqueous fluid will be consumed before the oil, and the oil will eventually pass through the aqueous reservoir to supply both inlet ports to the droplet forming features.

In order for the centrifuge system to function properly, in one embodiment, the tube is composed of polypropylene. In some embodiments, the insert is composed of polycarbonate or other material that would reproduce micrometer scale features.

What is claimed is:

1. A device for making an emulsion in a tube that utilizes centrifugal force for moving at least two fluids through droplet generating microfluidic pathways.

2. The device of claim 1, comprising at least a tube insert containing one fluid reservoir in fluidic communication with a droplet generation fluid pathway; and a capture tube.

3. The device of claim 1, wherein the first fluid is comprised of one or more PCR reagents, and the second fluid is an oil.

4. A method of emulsion PCR using the device as in claim 1.

5. The device of claim 1, wherein the device is disposable.

6. The device of claim 1, wherein the device is capable of producing droplets with a coefficient of variation of less than 50%.

7. The device of claim 1, wherein the device is capable of producing droplets with a coefficient of variation of less than 30%.

8. The device of claim 1, wherein the device is capable of producing droplets with a coefficient of variation of less than 10%.

9. The device of claim 1, wherein the device fits into a standard desktop centrifuge tube holder.

10. The device of claim 9, wherein the microfluidic plane is sloped at a 45 degree angle from the central vertical axis of the tube assembly.

11. The device of claim 1, further comprising one or more filter elements upstream of the microfluidic pathways.

12. The device as in claim 1 also containing a column.

13. The device as in claim 1 containing PCR reaction components including beads and reporter dyes.

14. The device as in claim 2 wherein the capture tube is also used for thermocycling.

15. The device as in claim 1, wherein the device is packaged in a kit for making droplets along with other reagents.

16. A process of making an emulsion using a device of claim 1, wherein the centrifugal speed is uniform throughout the droplet production phase of centrifugation.

17. A process of making an emulsion using a device of claim 1, wherein the centrifugal speed is varied during the droplet production phase of centrifugation.

18. A system for making an emulsion, comprising the device of claim 1; and a centrifuge.

19. The device of claim 2, wherein the first fluid reservoir does not reach the top of the tube insert, and is a partitioned volume of the second fluid reservoir.

20. The device of claim 2, wherein the first fluid reservoir reaches the top of the tube to effect complete separation of the two or more fluids.

21. A method of making an emulsion using a device as in claim 19, wherein the first reservoir is filled with a first fluid, and then a second fluid fills the second fluid reservoir, such that the second fluid covers the first.

22. The device of claim 2, wherein the tube is composed of polypropylene.
